(19) United States
(12) Patent Application Publication
(10) Pub. No.: US 2004/0043019 A1
(43) Pub. Date: Mar. 4, 2004

(54) Method of treating immune-mediated diseases by administration of IgM

(76) Inventors: Rauno Joks, Glen Cove, NY (US); Patricia Mueller, Miami, FL (US); Maja Nowakowski, Port Washington, NY (US); Hazel P. Drew, Laurelton, NY (US)

Correspondence Address: SCULLY SCOTT MURPHY & PRESSER, PC, 400 GARDEN CITY PLAZA, GARDEN CITY, NY, 11530

(21) Appl. No.: 10/438,118

(22) Filed: May 14, 2003

Related U.S. Application Data
(60) Provisional application No. 60/380,789, filed on May 15, 2002.

(51) Int. Cl. A61K 39/395

(52) U.S. Cl. 424/130.1

(57) ABSTRACT

Human polyclonal immunoglobulin M may be administered to patients suffering from a variety of immune-mediated diseases, including asthma, Common Variable Immunodeficiency (CVID), rheumatoid arthritis (RA), Systemic Lupus Erythmatosus (SLE), serum sickness and vasculitis, to treat the disease condition of those patients. Administration of immunoglobulin M results in a direct inhibitory effect on complement activity and a significant clinical improvement in the level of disease activity in patients with immune-mediated diseases.

### CROSS REFERENCE TO RELATED APPLICATION

This application claims the benefit of U.S. Provisional Application No. 60/380,789, filed on May 15, 2002.

### FIELD OF THE INVENTION

The present invention relates to the treatment of immune-mediated diseases, including autoimmune diseases, especially those resulting from immune complex formation and deposition. The invention relates to the treatment of asthma, Common Variable Immunodeficiency (CVID), rheumatoid arthritis (RA), and Systemic Lupus Erythmatosus (SLE). The invention also relates to the treatment of spondyloarthopathies including, inflammatory bowel disease, psoriatic arthritis, Reiter's Syndrome and ankylosing spondylitis, temporal arteritis, polymyalgia rheumatica and agammaglobulinemia. In accordance with the present invention immune-mediated diseases are treated by administration of a pharmaceutical composition comprising polyclonal IgM.

### BACKGROUND OF THE INVENTION

The present invention relates to a method for the treatment of immune-mediated diseases by administering a pharmaceutical composition comprising polyclonal IgM. Polyclonal IgM, and in particular human polyclonal IgM, is commonly obtained from commercial sources such as Chemicon International (Temecula, Calif.).

Intravenous immunoglobulin has been found to be of therapeutic value in a number of diseases. Its mechanism(s) of action are not fully elucidated. Miletic et al. (1996) J. Immunol. 156:749-757, suggested that its main effect is to serve as a preferred acceptor for the complement component C4 thereby preventing complement from binding to target tissues and inducing cellular damage. This group further found all immunoglobulin isotypes to have an inhibitory effect on C4 uptake.

One of the major mechanisms of damage resulting from immune complex formation in immune-mediated diseases such as SLE, is activation of the classical complement pathway with subsequent amplification of immune inflammation. Lupus flares are thought to be due to increased autoantibody formation with resultant increase in immune complex-mediated organ inflammation. Complement activity is known to be inherently related to immune-mediated disease. The role complement plays in asthma, for example, is presently being reevaluated. Complement split products, such as C3b, may promote the Th2 cytokine profile seen in asthma. Therefore, inhibitors of complement activity may be of benefit to both conditions.

Paradoxically, there are numerous cases documenting therapeutic benefit for multiple manifestations of SLE when patients are treated with pooled polyclonal human IgG antibodies in the form of intravenous immunoglobulin (IVIG) (Sherer et al. (1999) Clin. Rheumatol. 18:170-173; Welch et al. (1995) Arthritis Rheum. 38:1333-1337; Gordon, K. (1998) Lupus 7:429-433 and Ruiz-Valverde et al. (1994) 67:500-501). Numerous mechanisms have been proposed to explain this benefit. However, no single comprehensive theory can account for all the various clinical syndromes that improve from the administration of IVIG (Yu et al. (1999) New Eng. J. Med. 340:227-228).

In vitro work done by Frank and colleagues has demonstrated that immunoglobulins G, A, and M, can act to prevent complement-mediated cellular damage (Blood 77:376-380, 1991). However, despite the purported advances into the prevention of complement-mediated cellular damage, there remains a need to develop methods and compositions comprising IgM for the treatment of immune-mediated diseases.

### SUMMARY OF THE INVENTION

The present invention is directed to a method for treating an immune-mediated disease by administering a composition having polyclonal IgM in an amount sufficient to provide a clinically observable improvement in a patient's condition. The present invention is based on the discovery that polyclonal human IgM augmentation of serum in vitro results in decreased complement activity. Therefore, the administration of a composition containing immunoglobulin M to patients with immune-mediated disease results in a significant clinical improvement in the condition of the patient.

One object of the present invention provides a method of treating an immune-mediated disease in a patient by administering to the patient a composition comprising immunoglobulin M.

Another object of the invention provides a pharmaceutical composition comprising IgM and a pharmaceutically acceptable carrier.

Yet another object of the present invention provides a method of inhibiting complement activity in a patient suffering an immune-mediated disease by administering to said patient a composition comprising IgM.

### BRIEF DESCRIPTION OF THE DRAWINGS

FIG. 1 is a graph showing the percent decline in CH50 with IgM augmentation in CVID, RA, SLE and normal subjects.

FIG. 2 is a graph showing the percent decline in CH50 with IgM augmentation using increasing doses of IgM.

FIG. 3 is a bar graph showing an ELISA for detecting IgG immune complexes in vitro.

FIG. 4 is a bar graph showing an ELISA for detecting IgM immune complexes in vitro.

### DETAILED DESCRIPTION OF THE INVENTION

The present invention provides a method for treating a patient suffering from an immune-mediated disease, preferably a human patient, by administering a composition containing immunoglobulin M.

By “immune-mediated” disease is meant a pathogenic disease which triggers a characteristic immune response by cells including lymphocytes, antigen presenting cells and soluble mediators or cytokines produced by said cells. An immune-mediated disease manifests in symptoms such as pain and inflammation, for example, and includes such diseases as asthma, Common Variable Immunodeficiency (CVID), rheumatoid arthritis (RA), and Systemic Lupus Erythmatosus (SLE), for example. The methods of the present invention can be used to treat especially those immune-mediated disease resulting from immune complex formation and deposition, e.g., SLE, serum sickness and vasculitis.

A preferred composition for use in the present methods contains human immunoglobulin M, more preferably, human polyclonal IgM. Human polyclonal IgM is commonly obtained from a commercial source such as Chemicon International (Temecula, Calif.).

While not wishing to be bound by a particular mechanism of action or theory, the therapeutic effect of polyclonal IgM augmentation, in accordance with the present invention, apparently inhibits IgE responses.

An immunoglobulin composition useful in accordance with the present invention, contains human IgM purified by ammonium sulfate precipitation, G-200 chromatograpy and ion exchange chromatography.

The composition containing immunoglobulin M is administered in an amount sufficient to provide a clinically observable improvement in the disease symptoms of the patient. A “clinically observable improvement” when used herein refers to a significant subjective and/or objective remediation of symptoms associated with the patient's immune-mediated condition. For example, in the case of a patient suffering from rheumatoid arthritis, subjective remediation is characterized, in accordance with the present invention as including, but not limited to, tender joint(s), swollen joint(s) and stiffness reduction or amelioration assessments. Significant subjective remediation of symptoms denotes a patient's self-assessment or a physician=s assessment of stiffness, joint tenderness, swelling and the like. For example, an observable difference in swelling or tenderness in even one arthritic joint is significant. Absence of swelling or tenderness in a previously affected joint is most significant. Likewise, renewed freedom of movement in a joint(s) previously encumbered by an immune-mediated disease is significant. Objective measurements are also contemplated by the present invention, such as measurements total hemolytic complement activity based on determinations of a patient's CH50 values, for example.

In a preferred form of treating immune-mediated disease, the patient is administered a therapeutically effective amount of immunoglobulin M and a pharmaceutically acceptable carrier.

As used herein, a “pharmaceutically acceptable carrier” includes any and all solvents, dispersion media, coatings, antibacterial and antifungal agents, isotonic and absorption delaying agents, and the like. Some examples of substances which can serve as pharmaceutical carriers are sugars, such as lactose, glucose and sucrose; starches such as corn starch and potato starch; cellulose and its derivatives such as sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present. The use of such media and agents for pharmaceutically active substances is well known in the art. Except insofar as any conventional media or agent is incompatible with the active ingredients, its use in the therapeutic compositions is contemplated.

“Treating” or “treatment” as used herein means to ameliorate, suppress, mitigate or eliminate the clinical symptoms after the onset (i.e., clinical manifestation) of an immune-mediated disease, such as, for example, asthma. An effective or successful treatment provides a clinically observable improvement.

The compositions containing IgM may be administered intravenously, intramuscularly, intraperitoneally, orally or enterally, for example.

The dosage of the immunoglobulin compositions administered to the patient may be varied depending upon severity of the patient's condition and other clinical factors. Preferably, the dosage will be as small as possible while still providing a clinically observable and therapeutically effective result. The most preferable and therapeutically effective doses are those that have the largest effect in terms of alleviating the patient's disease condition; including pain or inflammation. Therapeutically effective dosages of the immunoglobulin M composition may range from as little as 0.1 mg/ml up to as much as 10 mg/ml per day.

Although the preferred dose is given in increments, it may also be given as a single dose. Further, the dose of the immunoglobulin M composition may be administered at any time during the day.

One practical advantage of the present invention is that the composition containing immunoglobulin M, may be administered in a convenient manner such as by the intravenous route, although the invention also contemplates administering of the claimed compositions by intramuscular, oral and intraperitoneal routes, for example. Intravenous administration is most preferred.

It is especially advantageous to formulate the immunoglobulin compositions of the present invention in dosage unit form for ease of administration and uniformity of dosage. “Dosage unit form” as used herein refers to physically discrete units suited as unitary dosages for the subjects to be treated, each unit containing a predetermined quantity of immunoglobulin M, calculated to produce the desired therapeutic effect in association with the required pharmaceutical carrier. The requirements for the dosage unit forms of the invention are dictated by and directly dependent on (a) the unique characteristics of the immunoglobulin M composition, and the particular therapeutic effect to be achieved, and (b) the limitations inherent in the art of formulating such an immunoglobulin composition for the treatment of immune-mediated disease herein disclosed in detail.

Another aspect of the present invention provides a pharmaceutical composition comprising immunoglobulin M and a pharmaceutically acceptable carrier.

A preferred pharmaceutical composition of the present invention contains human immunoglobulin M (IgM), more preferably, human polyclonal IgM.

Still another aspect of the present invention provides a method of inhibiting complement activity in a patient suffering an immune-mediated disease by administering to the patient a composition comprising IgM.

The invention will now be further described by the following non-limiting examples.

### EXAMPLE 1

Polyclonal human IgM augmentation of serum in vitro resulted in decreased complement activity, as measured by the CH50 assay. This effect was seen from serum from normal subjects and immune-complex disease patients, such as SLE and RA patients (See Table 1). This decrease in activity was not due to complement activation and consumption, as measured by a Clq-immune complex ELISA.

Specimen samples from subjects for CH50 determinations were frozen within one hour of collection and stored at −70° C. Purified, homogeneous, human IgM (Chemicon International) was lyophilized and added to the subject's serum in increasing IgM concentrations (0.1, 0.25, 0.5, 1.0 mg/ml). 200 μl of each subject's serum was incubated with varying amounts of IgM for 20 minutes at 37° C., including a duplicate sample without IgM. CR50 samples were determined by adding 100 μl of each specimen using a standard hemolytic assay protocol (Kent et al. (1963) Am J. Trop. Med. 12:103-116; Schur et al. (1968) Ann. Rev. Med. 19:1-24; Muller-Eberhard, H J (1988) 57:321-347; Ruddy, S. Complement: In Manual of Clinical Laboratory Immunology, 4^{th} ed. Rose, N R, De Macario, E C, Fahey J L, Friedman H, Penn G M, Eds. Am. Society for Microbiology, Wash. D.C. 1992, pp. 114-123, each of which are incorporated in their respective entireties by reference). A standard CH50 curve was generated by linear regression analysis. A reference CH50 value of 200 was used after scaling to compare CH50 test results over time.

TABLE 1
Subject's Initial Serum IgM Concentration (mg/ml)

| | | | | | | | | | | | |
|---|---|---|---|---|---|---|---|---|---|---|---|
| Subject's Initials | YM | RJ | NM | EP | JW | GP | BD | CL | IM | DF | DB |
| Diagnosis | Nml | Nml | Nml | CVID | CVID | SLE | SLE | SLE | RA | RA | RA |
| Serum IgM (mg/ml) | 2.36 | 0.8 | n/a | 0.43 | 0.32 | 0.93 | 1.14 | 0.96 | 1.02 | 2.48 | 1.86 |
| Normal range | | | | | | | | | | | |
| 0.6-2.6 mg/ml | | | | | | | | | | | |

### EXAMPLE 2

CIC-Clq Enzyme Immunoassay (Quidel) was used to determine presence of both IgG and IgM immune complexes. A solid phase human Clq purified protein in microtiter wells was incubated with CH50 assay (supernatants which had been immediately frozen at −70° C after the hemolytic assay). Clq binds to the Fc portion of immune complex IgG. Following the protocol provided by the manufacture, horseradish peroxidase (HRP)-conjugated goat anti-human IgG was added to each well. During incubation, the conjugate bound to IgG immune complexes which were bound to the Clq-coated microassay wells. Appropriate chromogenic substrate was added to each well to produce a color reaction. The test and standard specimens were measured spectrophotometrically (Å405 values). In order to detect IgM immune complexes, an HRP-conjugated goat anti-human IgM (1:10, 000) was added instead of anti-IgG.

Results

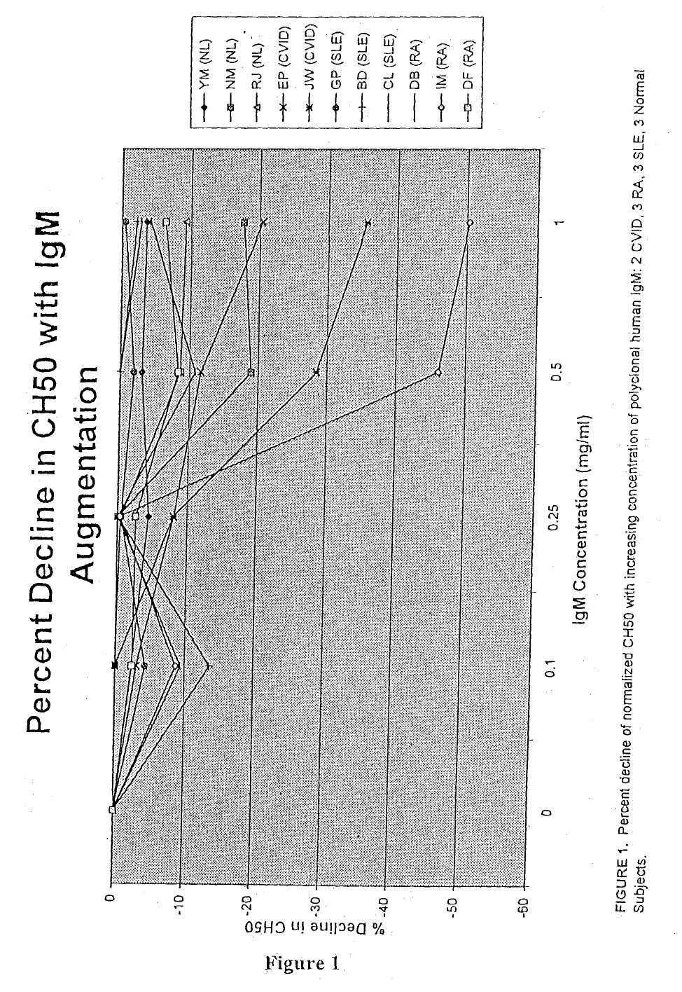

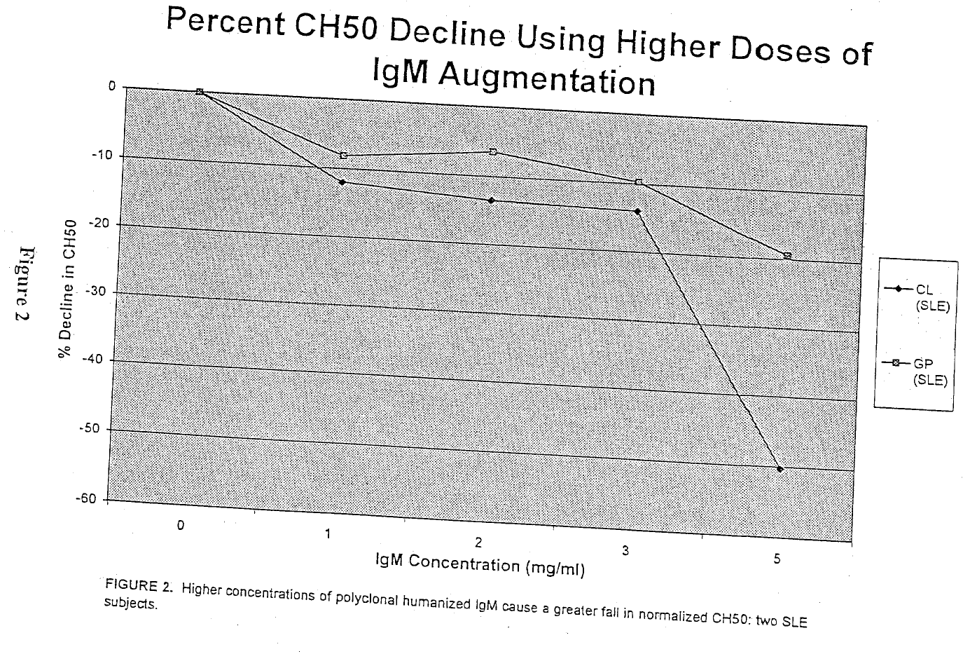

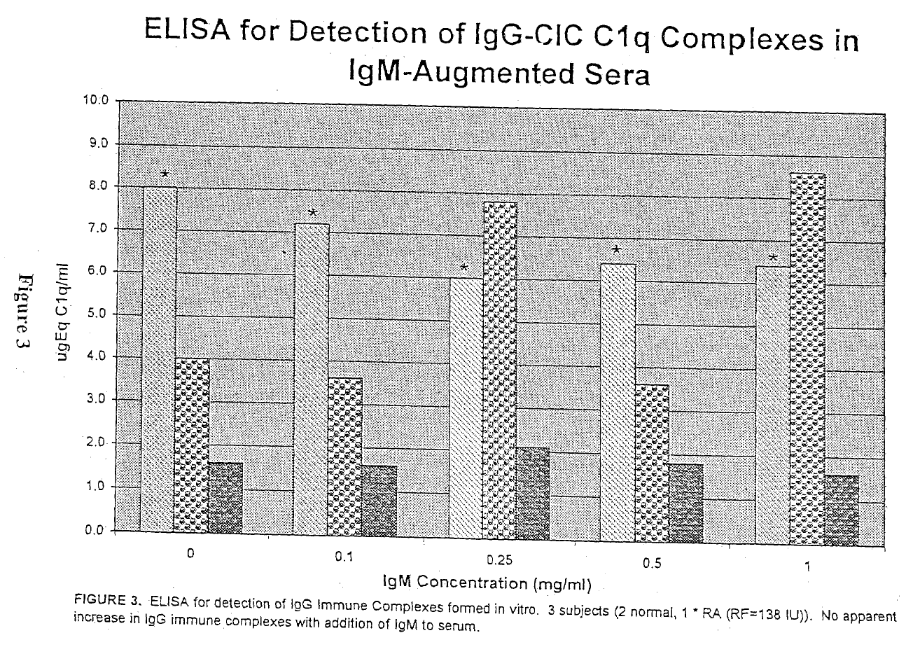

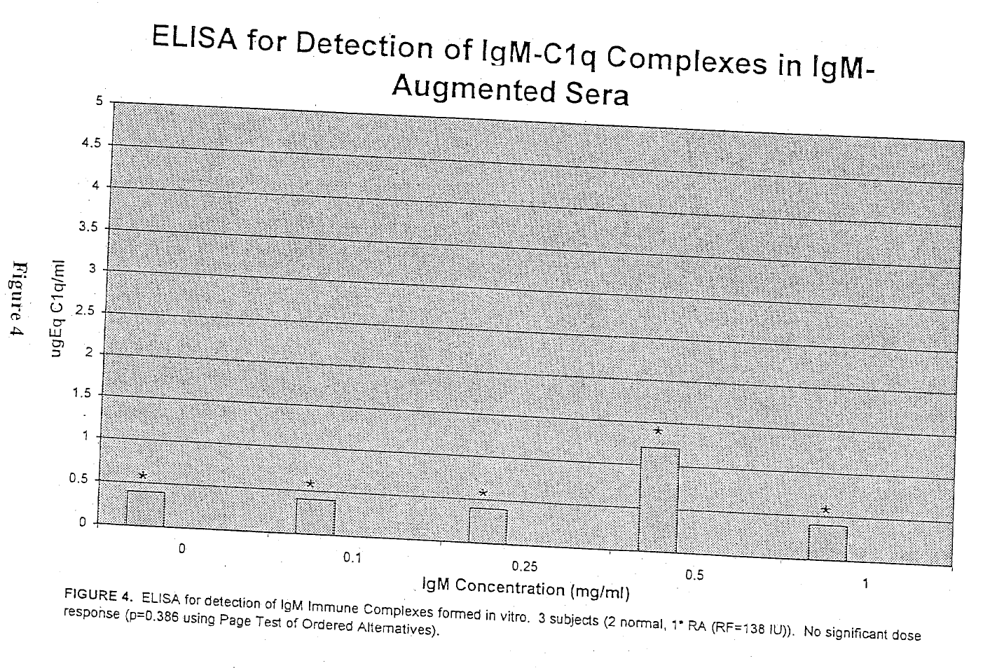

Addition of polyclonal human IgM in varying amounts to human serum resulted in a significant dose-dependent decrease in CH50 levels (FIG. 1). The decrease in complement activity was seen in all of the states of altered immunity assayed (CVID, RA, SLE) as well as in normal serum. Maximum decrease in complement activity to date was found in SLE serum (50% decrease) with 5 mg/ml IgM augmentation (FIG. 2). There was no evidence of in vitro IgG or IgM immune complex formation as detected by solid phase CIC-Clq assay (FIGS. 3 and 4).

Discussion

The results demonstrate that IgM augmentation of serum from subjects with SLE, RA, Common Variable Immunodeficiency (CVID), and healthy controls affected total serum complement activity as measured by the CH50 complement hemolytic activity assay. The effects of polyclonal human IgM on functional complement activity in diseases of altered humoral immunity were assessed. IgM was added, in varying amounts (0.1, 0.25, 0.5, 1.0 mg/ml), to the sera of patients with common variable immunodeficiency (CVID), rheumatoid arthritis (RA) and healthy controls. Total hemolytic complement activity was immediately determined using the CH50 assay.

The present invention demonstrates, for the first time, a dose-dependent decline in CH50, with a maximum decrease of about 20% of complement activity, in all three groups (p<0.01 vs. untreated sera). A Clq solid phase ELISA was performed to determine whether the fall in complement activity was due to formation of in vitro IgM immune complexes with subsequent complement activation. There was no evidence of IgM induced complex formation using this assay (p=ns). These results demonstrate a direct inhibitory effect of IgM on complement activity. The decrease in CH50 values apparently results from either of the two processes: (a) complement consumption resulting from in vitro immune complex formation, or (b) inhibition of complement factor usage in the hemolytic process. Although immune complex formation was only assayed with a single assay, the invention demonstrates an inhibition of complement activity resulting from IgM augmentation.

What is claimed is:

1. A method of treating an immune-mediated disease in a patient comprising administering to said patient a composition comprising immunoglobulin M and a pharmaceutically-acceptable carrier.

2. The method of claim 1, wherein said immune-mediated disease is selected from the group consisting of asthma, Common Variable Immunodeficiency (CVID), rheumatoid arthritis (RA), Systemic Lupus Erythmatosus (SLE), serum sickness and vasculitis.

3. The method of claim 1, wherein said immunoglobulin M is human immunoglobulin M.

4. The method of claim 3, wherein said human immunoglobulin M is polyclonal immunoglobulin.

5. The method of claim 1, wherein said composition is administered in an amount sufficient to provide a clinically observable improvement in the disease symptoms of said patient.

6. The method of claim 5, wherein said immune-mediated disease results from immune complex formation and deposition, and said improvement in the disease symptoms is measured by a reduction of total hemolytic complement activity in the serum of said patient.

7. The method of claim 1, wherein said composition is administered intravenously, intramuscularly, intraperitoneally, orally or enterally.

8. A method of inhibiting complement activity in a patient suffering an immune-mediated disease comprising administering to the patient a composition comprising IgM and a pharmaceutically acceptable carrier.

9. The method of claim 8, wherein said immune-mediated disease is selected from the group consisting of Common Variable Immunodeficiency (CVID), rheumatoid arthritis (RA), Systemic Lupus Erythmatosus (SLE), serum sickness and vasculitis.

10. The method of claim 8, wherein said immunoglobulin M is human immunoglobulin M.

11. The method of claim 10, wherein said human immunoglobulin M is polyclonal immunoglobulin.

12. The method of claim 8, wherein said composition is administered intravenously, intramuscularly, intraperitoneally, orally or enterally.

13. A pharmaceutical composition comprising immunoglobulin M and a pharmaceutically acceptable carrier, wherein said immunoglobulin M is in an amount sufficient to provide a clinically observable improvement in a patient suffering an immune-mediated disease.

14. A pharmaceutical composition comprising immunoglobulin M and a pharmaceutically acceptable carrier, wherein said immunoglobulin M is in an amount sufficient to inhibit complement activity in a patient suffering an immune-mediated disease.

15. The composition of claim 13 or 14, wherein said human immunoglobulin M is human polyclonal IgM.

16. The composition of claim 13 or 14, wherein said immune-mediated disease is selected from the group consisting of asthma, Common Variable Immunodeficiency (CVID), rheumatoid arthritis (RA), Systemic Lupus Erythmatosus (SLE), serum sickness and vasculitis.
